(19) United States
(12) Patent Application Publication
(10) Pub. No.: US 2026/0269080 A1
(43) Pub. Date: Sep. 10, 2026

(54) METHOD FOR PROVIDING EXPLANATION FOR PATIENT STATE PREDICTION AND ELECTRONIC APPARATUS THEREFOR

(71) Applicant: AITRICS CO., LTD., Seoul (KR)

(72) Inventors: Changhun KIM, Seoul (KR); Sangchul HAHN, Seoul (KR); Kwang Joon KIM, Seoul (KR)

(73) Assignee: AITRICS CO., LTD., Seoul (KR)

(21) Appl. No.: 19/674,825

(22) Filed: May 12, 2026

Related U.S. Application Data
(63) Continuation of application No. PCT/KR2025/017743, filed on Oct. 31, 2025.

(30) Foreign Application Priority Data
Jan. 22, 2025 (KR) 10-2025-0009664

(51) Int. Cl. G16H 50/30; G16H 10/60

(52) U.S. Cl. CPC G16H 50/30; G16H 10/60

(57) ABSTRACT

A method for providing an explanation for patient state prediction and an electronic apparatus therefor are disclosed. The method includes acquiring time-series data on a patient, acquiring, from an artificial intelligence-based prediction model, a prediction result that is output by the prediction model based on the time-series data, acquiring, from an explainable artificial intelligence-based explanation model, explanation data that is output by the explanation model based on the time-series data and the prediction result, inputting input data including the time-series data, the prediction result, and the explanation data to an artificial intelligence-based language model, and acquiring, from the language model, natural language text data that is output by the language model based on the input data and includes a reasoning basis of the prediction model for the prediction result.

### CROSS-REFERENCE TO RELATED APPLICATION(S)

This application is a Continuation of International Patent Application No. PCT/KR2025/017743, filed on Oct. 31, 2025, which claims the benefit of Korean Patent Application No. 10-2025-0009664, filed on Jan. 22, 2025, in the Korean Intellectual Property Office, the disclosure of which is incorporated herein by reference.

### BACKGROUND

### 1. Field of the Invention

The present disclosure relates to a technology that provides an explanation for patient state prediction by using an electronic apparatus. More specifically, the present disclosure relates to a technology that explains a reasoning basis of a prediction model for a result of predicting a patient state.

### 2. Description of the Related Art

As an artificial intelligence model is currently used to predict the state of a patient, the speed and efficiency of diagnosis is significantly increased. By supplementing insufficient medical personnel and substantially reducing the heavy workload of a medical institution, a more comfortable clinical environment may be provided to both medical staff and patients.

Specifically, an artificial intelligence model utilized for the prediction and measurement of a major adverse events score (MAES), a sepsis score, a mortality score, and the like, as an artificial intelligence model utilized lately in a medical environment, may include a wide range of approaches from a conventional statistical model to an up-to-date deep learning-based technique. These risk prediction models take, as an input, an electronic health record (EHR), clinical test data, a biosignal, a prescription record, imaging, and biodata extracted from a wearable device, calculate a probability or score indicating that a patient may experience a major adverse event, sepsis, mortality, or the like at a predetermined time point or during hospitalization period, and provide the probability or score to medical personnel to reduce the workload. However, such an artificial intelligence model fundamentally is “black boxes” of which the decision-making process is difficult for humans to interpret. Thus, neither the medical staff nor the patient may grasp a basis for prediction by the model. Accordingly, the patient may not trust diagnosis by the model, and even when the model gives a diagnosis indicating that medication, surgery, or the like is required, the patient may hesitate to comply.

Furthermore, while the requirements of various regulations in the medical field are to be satisfied based on a clear understanding and evidentiary basis for a diagnosis in some cases, a basis for the diagnosis from the artificial intelligence model may not be identified. Accordingly, a clinical approval may be denied due to the regulation.

Additionally, the artificial intelligence model may have the risk of bias in the process of learning. When the basis for the diagnosis by the model may not be explained, identifying such a bias accurately and removing the risk may become greatly difficult.

### SUMMARY

An aspect aims to gain trust of a patient in diagnosis by an artificial intelligence model by providing a reasoning basis of the model in a form of natural language text.

Furthermore, another aspect aims to enhance reliability of the reasoning basis provided by the model by utilizing various types of additional information that assist in inferring the reasoning basis of the artificial intelligence model.

The technical objectives to be achieved by the present disclosure are not limited to the technical objectives mentioned above, and other technical objectives may be inferred from the following example embodiments.

According to an aspect, there is provided A method for providing an explanation for patient state prediction through an electronic apparatus, the method including acquiring time-series data on a patient, acquiring, from an artificial intelligence-based prediction model, a prediction result that is output by the prediction model based on the time-series data, acquiring, from an explainable artificial intelligence-based explanation model, explanation data that is output by the explanation model based on the time-series data and the prediction result, inputting input data including the time-series data, the prediction result, and the explanation data to an artificial intelligence-based language model, and acquiring, from the language model, natural language text data that is output by the language model based on the input data and includes a reasoning basis of the prediction model for the prediction result.

The time-series data may include information on a time at which a biosignal of the patient is measured, a value of the biosignal, and a type of the biosignal, the prediction result may include at least a portion of a sepsis score, an acute major adverse events score, and an acute mortality score of the patient, and the explanation data may include an input attribution score of the time-series data for the prediction result.

The inputting may include generating input data corresponding to an input format of the language model by concatenating the time-series data, the prediction result, and the explanation data.

The natural language text data may include, as the reasoning basis of the prediction model, a biosignal identified by the explanation data among a plurality of biosignals forming the time-series data.

The method may further include acquiring abnormality data in which an abnormality of at least a portion of biosignals among a plurality of biosignals forming the time-series data is evaluated based on a rule, and the input data may further include the abnormality data, and the natural language text data may include, as the reasoning basis of the prediction model, at least a portion of a biosignal identified by the explanation data and a biosignal determined to be abnormal according to the abnormality data among the plurality of biosignals forming the time-series data.

The abnormality data may include whether the at least a portion of biosignals among the plurality of biosignals forming the time-series data is normal or abnormal, a normal range value for the at least a portion of biosignals, and a class value classifying an abnormality of a biosignal determined to be abnormal.

The natural language text data may include a biosignal identified by the explanation data and simultaneously determined to be abnormal according to the abnormality data as a first-priority reasoning basis of the prediction model, a biosignal identified by the explanation data and not determined to be abnormal according to the abnormality data as a second-priority reasoning basis of the prediction model, and a biosignal determined to be abnormal according to the abnormality data and not identified by the explanation data as a third-priority reasoning basis of the prediction model.

The method may further include acquiring electronic health record (EHR) data including a past medical history and a treatment history of the patient, and the input data may further include the EHR data, and the natural language text data may include, as the reasoning basis of the prediction model, at least a portion of a biosignal identified by the explanation data and a biosignal identified by the EHR data among a plurality of biosignals forming the time-series data.

When the biosignal identified by the EHR data corresponds to the reasoning basis of the prediction model, the natural language text data may further include text information that explains the past medical history and the treatment history of the patient as a basis for determining a symptom of the patient and explains a diagnosis content corresponding to the biosignal identified by the EHR data as a result of determining the symptom of the patient.

The method may further include acquiring a result of analysis by a specialist on the time-series data and the prediction result, and the input data may further include the result of analysis by the specialist, and the natural language text data may include, as the reasoning basis of the prediction model, at least a portion of a biosignal identified by the explanation data and a biosignal identified by the result of analysis by the specialist among a plurality of biosignals forming the time-series data.

According to another aspect, there is also provided an electronic apparatus for providing an explanation for patient state prediction, the electronic apparatus including a transceiver configured to communicate with an external entity, a memory configured to store an instruction, and a processor, and the processor is configured to control the transceiver and the memory to acquire time-series data on a patient, acquire, from an artificial intelligence-based prediction model, a prediction result that is output by the prediction model based on the time-series data, acquire, from an explainable artificial intelligence-based explanation model, explanation data that is output by the explanation model based on the time-series data and the prediction result, input input data including the time-series data, the prediction result, and the explanation data to an artificial intelligence-based language model, and acquire, from the language model, natural language text data that is output by the language model based on the input data and includes a reasoning basis of the prediction model for the prediction result.

Details of other example embodiments are included in the detailed description and the drawings.

According to example embodiments, it is possible to provide a reasoning basis of an artificial intelligence model in natural language text and thus enhance trust of medical staff and a patient in diagnosis by the model and improve an ability to explain a diagnostic result.

According to example embodiments, it is possible to provide a personalized medical service by comprehensively reflect a degree of abnormality, a past medical history of the patient, a treatment history of the patient, a specialist opinion, and the like in a prediction process of the model and facilitate smooth communication between the patient and the medical staff.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a diagram schematically illustrating a system providing an explanation for patient state prediction according to the present disclosure;

FIG. 2 is a flowchart illustrating a method for providing an explanation for patient state prediction according to an example embodiment;

FIGS. 3 through 6 are example diagrams illustrating that input data is input to a language model and natural language text data is output; and

FIG. 7 is a block diagram illustrating an electronic apparatus according to various example embodiments.

### DETAILED DESCRIPTION

Hereinafter, detailed example embodiments will be described with reference to the accompanying drawings. The following detailed description is provided to assist in the comprehensive understanding of methods, apparatuses, and/or systems described herein. However, it is merely an example, and the disclosed example embodiments are not limited thereto.

In describing the example embodiments, when the detailed description of related known technologies is determined to unnecessarily obscure the gist of the disclosed example embodiments, the detailed description will be omitted. In addition, terms described below are defined in consideration of functions in the disclosed example embodiments and may vary depending on intentions or customs of users or operators. Therefore, the definitions thereof should be made based on the contents throughout the present specification. Terms used in the detailed description are merely for describing the example embodiments and is not to be limitative. Unless clearly used otherwise, a term in the singular form includes the term in the plural form. In the present description, terms such as “comprise” or “include” are intended to indicate characteristics, numbers, steps, operations, elements, or a portion or combination thereof and is not be construed as excluding the existence or possibility of one or more other characteristics, numbers, steps, operations, elements, or a portion or combination thereof other than those described.

The terms used in the example embodiments are selected from currently widely used general terms in consideration of functions in the present disclosure, but these may vary depending on the intentions of technicians working in the field, precedents, the emergence of new technologies, and the like. In addition, in some cases, there are terms arbitrarily selected by the applicant, and in this case, the meanings thereof will be described in detail in the corresponding description part. Therefore, the terms used in the present disclosure should be defined based on the meanings of the terms and the contents throughout the present disclosure, rather than simple names of the terms.

Throughout the specification, when a part “comprises” an element, unless specifically described otherwise, it means that the part may further include another element, rather than exclude another element. In addition, terms such as “ . . . part” and “ . . . module” described in the specification refer to a unit that processes at least one function or operation, which may be implemented as hardware or software or as a combination of hardware and software and may not be clearly distinguished in an operation unlike the illustrated examples.

The term “at least one of a, b, and c” described throughout the specification may encompass ‘a alone’, ‘b alone’, ‘c alone’, ‘a and b’, ‘a and c’, ‘b and c’, or ‘all of a, b, and c’.

A “terminal” described below may be implemented as a computer or a portable terminal capable of accessing a server or another terminal through a network. Here, the computer includes, for example, a notebook computer, a desktop computer, or a laptop computer equipped with a web browser, and the portable terminal includes, for example, a wireless communication device ensuring portability and mobility, such as a terminal based on communication such as International Mobile Telecommunication (IMT), code division multiple access (CDMA), W-code division multiple access (W-CDMA), or long term evolution (LTE), and all kinds of handheld-based wireless communication devices such as a smartphone and a tablet PC.

In the following description, terms such as the “transmission,” “communication,” “sending,” or “receiving” of a signal or information and other terms with similar meanings include not only the direct transmission of the signal or information from one element to another element but also the transmission through another element.

In particular, “transmitting” or “sending” a signal or information to an element indicates the final destination of the signal or information and does not mean a direct destination. This is the same for the “receiving” of the signal or information. In addition, in the present specification, that two or more pieces of data or information are “associated” means that when one piece of data (or information) is acquired, at least a portion of other data (or information) may be acquired based thereon.

In addition, terms such as first and second may be used to describe various elements, but the elements should not be limited by the terms. The terms may be used for the purpose of distinguishing one element from another element.

For example, without departing from the scope of the present disclosure, a first component may be referred to as a second component, and similarly, the second component may be also referred to as the first component.

In describing the example embodiments, the description of technical content well known in the technical field to which the present disclosure belongs and not directly related to the present disclosure will be omitted. This is to omit an unnecessary description so as to more clearly convey the gist of the present disclosure without obscuring the same.

For the same reason, some elements are exaggerated, omitted, or schematically illustrated in the accompanying drawings. In addition, the size of each element does not fully reflect the actual size. The same reference numerals are assigned to the same or corresponding elements in each drawing.

Advantages and features of the present disclosure and methods of achieving the same will become apparent with reference to the example embodiments described in detail below together with the accompanying drawings. However, the present disclosure is not limited to the example embodiments disclosed below but may be implemented in various different forms, and the present example embodiments are merely provided to complete the present disclosure and to fully inform those skilled in the art of the scope of the present disclosure, and the present disclosure is defined only by the scope of the claims. The same reference numerals refer to the elements throughout the specification.

It will be understood that each block of the flowchart illustrations and the combinations of the flowchart illustrations may be performed by computer program instructions. Since these computer program instructions may be mounted on a processor of a general-purpose computer, a special-purpose computer, or another programmable data processing device, the instructions executed through the processor of the computer or other programmable data processing device may create means for performing functions described in the flowchart block(s). Since these computer program instructions may be also stored in a computer-usable or computer-readable memory that may direct the computer or other programmable data processing device in order to implement a function in a particular manner, the instructions stored in the computer-usable or computer-readable memory may produce an article of manufacture including instruction means which perform the function described in the flowchart block(s). Since the computer program instructions may be also mounted on the computer or other programmable data processing device, a series of operational steps may be performed on the computer or other programmable data processing device to generate a computer-executed process, so that steps for executing the functions described in the flowchart block(s) may be provided.

In addition, each block may represent a module, segment, or portion of code including one or more executable instructions for implementing identified logical function(s). It should also be noted that in some alternative implementations, the functions described in the blocks may occur out of order. For example, two blocks shown in succession may in fact be executed substantially concurrently, or the blocks may sometimes be executed in the reverse order, depending on the corresponding functions.

Hereinafter, the example embodiments of the present disclosure will be described in detail with reference to the accompanying drawings so that those skilled in the art to which the present disclosure belongs may easily carry out the present disclosure. However, the present disclosure may be implemented in various different forms and is not limited to the example embodiments described herein.

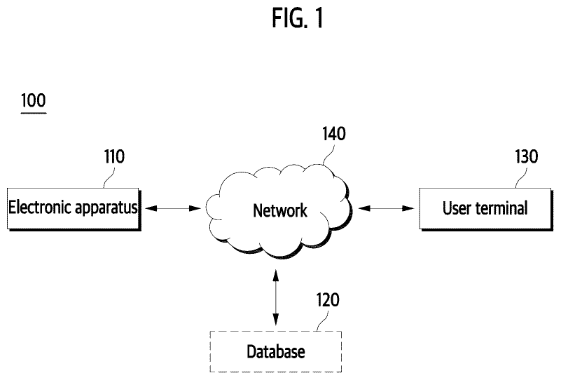

FIG. 1 is a diagram schematically illustrating a system providing an explanation for patient state prediction according to the present disclosure. Those skilled in the art related to the present example embodiment will understand that other general-purpose elements may be further included in addition to elements illustrated in FIG. 1.

Referring to FIG. 1, the system includes an electronic apparatus 110, a database 120, a user terminal 130, and a network 140. The electronic apparatus 110, the database 120, and the user terminal 130 may communicate with each other through a network. The network includes a local area network (LAN), a wide area network (WAN), a value added network (VAN), a mobile radio communication network, a satellite communication network, and a combination thereof, is a data communication network in a comprehensive sense that allows smooth communication between each system entity illustrated in FIG. 1, and may include a wired Internet, a wireless Internet, and a mobile wireless communication network. Wireless communication may include, for example, wireless LAN (e.g., Wi-Fi), Bluetooth, Bluetooth Low Energy, Zigbee, Wi-Fi Direct (WFD), ultra-Wideband (UWB), Infrared Data Association (IrDA), and near field communication (NFC), but it is merely an example.

The electronic apparatus 110 is an apparatus that explains, with respect to a patient state predicted by an artificial intelligence model, a reason for the model predicting such a result. To this end, the electronic apparatus 110 may acquire information from various sources. For example, the electronic apparatus 110 may acquire data used by the artificial intelligence model for prediction (reasoning) from a database of a medical institution and may acquire a prediction result of the artificial intelligence model from a cloud server providing a service of the model. In addition, since data explaining a decision-making process of the model may be generated through a separate entity that analyzes a decision logic of the model, the electronic apparatus 110 may acquire such data from the entity. Although FIG. 1 illustrates that the electronic apparatus 110 receives information from one database 120 through the network 140, this is for convenience of description, and the number and types of sources from which the electronic apparatus 110 acquires information may vary according to example embodiments.

Meanwhile, the electronic apparatus 110 may generate information for explaining a prediction basis of the model and store the generated information in the electronic apparatus 110 or in a database or a cloud server linked with the electronic apparatus 110. At this point, the electronic apparatus 110 may tag the generated information with an identifier corresponding to a type of the prediction result of the model, a medical institution using the model, or a user account requesting the prediction by the model and store the tagged information, so that a database that allows an easy inquiry according to the type of the prediction result, the medical institution, or the user may be created.

In addition, the electronic apparatus 110 may generate the information for explaining the prediction basis of the model and provide the information to the user terminal 130 through the network 140. The user terminal 130 is a terminal of a user account using a service provided by the electronic apparatus 110 or a service provided by the model and may be, for example, a computer of medical staff working in the medical institution or a smartphone of a patient hospitalized in the medical institution, but it is merely an example. The electronic apparatus 110 may generate the information for explaining the prediction basis of the model in response to a request from the user terminal 130 and transmit the generated information to the user terminal 130 which has transmitted the request.

According to an example embodiment, the user terminal 130 to which the electronic apparatus 110 transmits the generated information may be a terminal separate from a device that has transmitted the request. For example, the electronic apparatus 110 may generate the information for explaining the prediction basis of the model at a request of a doctor (e.g., an administrator account) and transmit the generated information to the user terminal 130 to which a patient account is connected for logging in. In this regard, when a terminal requesting generation of the information is different from a terminal to which the information is to be transmitted, or when the terminal to which the information is to be transmitted is not a pre-registered terminal (e.g., a terminal of the medical institution), the electronic apparatus 110 may encrypt the generated information with a private key of the electronic apparatus 110 and transmit a public key of the electronic apparatus 110 together with the generated information to a terminal to receive the information. This is to prevent the information generated by the electronic apparatus 110 from being leaked to an unexpected third party, because the information generated by the electronic apparatus 110 may be sensitive information including a result of predicting the patient state and the basis therefor.

Meanwhile, the electronic apparatus 110 may generate the information for explaining the prediction basis of the model, process the information in response to a request from the user terminal 130, and then transmit the processed information to the user terminal 130. As an example, in response to the request from the user terminal 130, the electronic apparatus 110 may apply a graphic effect (e.g., highlight) or a font effect (e.g., bold font or changing a font size) to a portion corresponding to the prediction basis of the model in the generated information. As another example, for a user who is not a skilled person such as medical staff, the electronic apparatus 110 may translate a word written in English into Korean in the generated information or convert a technical term into a general term in response to the request from the user terminal 130. As still another example, the electronic apparatus 110 may summarize the generated information into a predetermined number of characters or less in response to the request from the user terminal 130. This is to prevent an excessive amount of information from being provided to medical staff that is to diagnose many patients within a limited time.

In this regard, a more detailed description will be provided below with reference to the accompanying drawings.

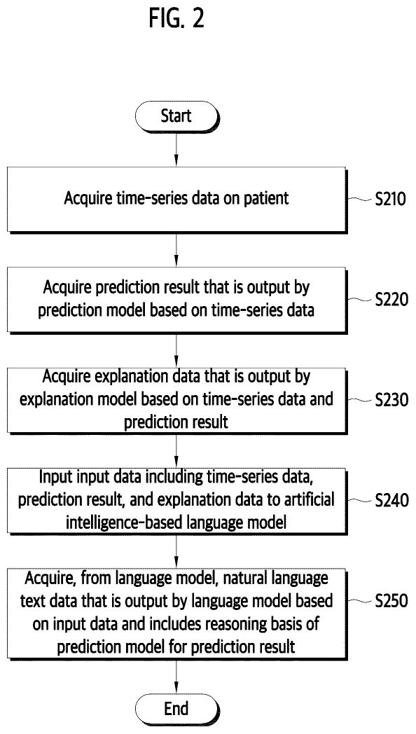

FIG. 2 is a flowchart illustrating a method for providing an explanation for patient state prediction according to an example embodiment. The method illustrated in FIG. 2 is described as being performed by the electronic apparatus 110 described above, but it is merely an example, and the method illustrated in FIG. 2 may be performed by another apparatus or by a combination of the electronic apparatus 110 and a separate apparatus.

In operation S210, the electronic apparatus 110 may acquire time-series data on a patient.

According to an example embodiment, the time-series data on the patient may include information on a time at which a biosignal of the patient is measured, a value of the biosignal, and a type of the biosignal.

In an example embodiment, the electronic apparatus 110 may acquire the entire time-series data on the patient from one device, server, or database. Alternatively, depending on a type of the time-series data, the electronic apparatus 110 may acquire a portion of the time-series data from a digital device that measures data by contacting a body of the patient or a device assisting the digital device (hereinafter, such devices are collectively referred to as a ‘first device’) and acquire a remainder of the time-series data from a device that stores medical information recorded, measured, or analyzed for the patient (hereinafter, such a device is collectively referred to as a ‘second device’). The electronic apparatus 110 may access an Internet of Things (IoT) application to acquire data from the first device. At this point, a network used for the electronic apparatus 110 and the first device to access the IoT application may be the same as or different from a network used for the electronic apparatus 110 to communicate with the second device.

According to an example embodiment, the time-series data may include the information on the time at which the biosignal of the patient is measured, the value of the biosignal, and the type of the biosignal. The electronic apparatus 110 may generate the time-series data by acquiring the time at which the biosignal of the patient is measured, the value of the biosignal, and the type of the biosignal and then inputting the time, the value, and the type to a template corresponding to the time-series data.

In operation S220, the electronic apparatus 110 may acquire, from an artificial intelligence-based prediction model, a prediction result that is output by the prediction model based on the time-series data.

According to an example embodiment, the prediction model may include a large language model (LLM) based on a transformer architecture. For example, the prediction model may analyze and tokenize an input in a text form, and a self-attention mechanism may be applied thereto.

In addition, the prediction result output by the prediction model may include information on a probability that an abnormality in a state to be predicted of the patient occurs within a time period from a current time point to a prediction time point. For example, the prediction result output by the prediction model may include at least a portion of a sepsis score of the patient, an acute major adverse events score, and an acute mortality score.

In operation S230, the electronic apparatus 110 may acquire, from an explainable artificial intelligence (XAI)-based explanation model, explanation data that is output by the explanation model based on the time-series data and the prediction result.

In an example embodiment, the explanation data output by the explanation model may include an input attribution score of the time-series data for the prediction result output by the prediction model. Specifically, the XAI-based explanation model may indicate what feature included in the time-series data has contributed to output of the prediction result by the prediction model and a degree to which the feature has contributed, as an input attribution score of each feature included in the time-series data.

As an example, an explanation model based on a SHapley Additive explanations (SHAP) technique uses a Shapley value of cooperative game theory. The input attribution score of each feature for prediction may be calculated by considering all possible subsets of features including or excluding a corresponding feature. The explanation model based on the SHAP technique may assume that the prediction result is calculated by adding up a baseline value (which refers to an output of the prediction model for a neutral input such as an average prediction for all inputs) and a sum of respective input attribution scores of features, as shown in the following Equation 1, may evaluate how the prediction result changes when each feature is included or excluded in an input to the prediction model, and may calculate a difference between prediction results. In Equation 1, ƒ(x) denotes the prediction result, φ₀ denotes the baseline value, and φᵢ denotes an input attribution score of feature i.

$$f ( x ) = {ϕ}_{0} + {\sum }_{i = 1}^{n} {ϕ}_{i} \qquad [ Equation 1 ]$$

As another example, an explanation model based on a Local Interpretable Model-agnostic Explanations (LIME) technique may explain the prediction result by partially approximating the prediction model by using an interpretable surrogate model (typically, a linear model). Specifically, the explanation model focuses on a surrounding region of a predetermined feature (input) and approximates a complex prediction model by using a simple model. To this end, the explanation model generates new data by masking a portion of the input to the prediction model, adding noise to the input, or the like and assigns higher a weight to a point closer to the original input by using a kernel function. The surrogate model is trained through the input (data set) to which the weight is applied, and a coefficient of the surrogate model may show the input attribution score for each feature.

In operation S240, the electronic apparatus 110 may input input data including the time-series data, the prediction result, and the explanation data to an artificial intelligence-based language model.

In an example embodiment, the electronic apparatus 110 may generate input data corresponding to an input format of the language model by concatenating the time-series data, the prediction result, and the explanation data. As another example embodiment, the electronic apparatus 110 may generate the input data by tagging each of the time-series data, the prediction resudisclosurelt, and the explanation data with a label, assigning an identifier thereto, or adding a subtitle in natural language so that the language model may distinguish between the time-series data, the prediction result, and the explanation data.

In an example embodiment, the language model may be a model trained to output data in a form of natural language text based on input data formed only with a numerical value or input data further including a character other than the numerical value. For example, the language model may be a model to which a Natural Language Explanation (NLE) technique for explaining a basis for prediction by an artificial intelligence model is applied and may include an LLM based on the transformer architecture for this purpose. However, the language model does not have the same structure as the prediction model, and a detailed architecture thereof may differ depending on example embodiments. The language model may include a Text-To-Text Transfer Transformer (T5) model or an explainable GPT model and may be configured with a plurality of sub-models.

In operation S250, the electronic apparatus 110 may acquire, from the language model, natural language text data that is output by the language model based on the input data and includes a reasoning basis of the prediction model for the prediction result.

In an example embodiment, the language model may be trained to output the natural language text data which includes, as the reasoning basis of the prediction model, a biosignal identified by the explanation data among a plurality of biosignals forming the time-series data.

In an example embodiment, the natural language text data output by the language model by receiving the time-series data, the prediction result, and the explanation data may include text data having a descriptive sentence structure associated with the plurality of biosignals included in the time-series data. In the text data, a graphic effect (for example, highlight) or a font effect (for example, bold font or changing a font size) may be applied to the biosignal identified by the explanation data. When providing the natural language text data to the user terminal 130, the electronic apparatus 110 may translate a word written in English into Korean or convert a technical term into a general term for a user who is not a skilled person such as medical staff. As another example, the electronic apparatus 110 may summarize the natural language text data into a predetermined number of characters or less. Both the natural language text data before conversion or summarization and the natural language text data after conversion or summarization may be provided on a screen of the user terminal 130, and whether to convert or summarize the natural language text data may be switched at a request of the user terminal 130 to the electronic apparatus 110.

Meanwhile, although FIG. 2 illustrates that the electronic apparatus 110 performs operations S210 to S230 separately and sequentially, but this is for convenience of description. According to an example embodiment, the electronic apparatus 110 may perform operations S210 to S230 simultaneously or may separately perform some of operations S210 to S230 while changing an order thereof.

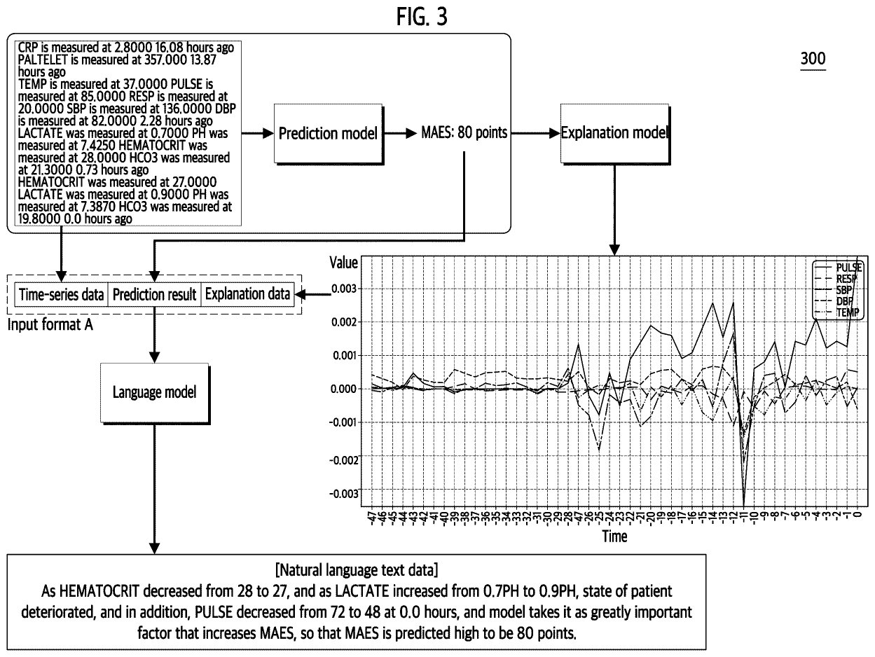

FIGS. 3 through 6 are example diagrams illustrating that input data is input to a language model and natural language text data is output.

FIG. 3 describes an example in which the input data including time-series data, a prediction result, and explanation data is input to the language model and the natural language text data is output.

The time-series data of FIG. 3 may include a numerical value for various types of biosignals measured at a predetermined time before a current time. For example, data stating “CRP was measured at 2.8000 16.08 hours ago” indicates that a C-reactive protein (CRP) level has been measured at 2.8000 16.0833 hours before the current time.

According to an example embodiment, the electronic apparatus 110 may sort an order of the time-series data before inputting the time-series data to an input format A. As an example, the electronic apparatus 110 may sort each row of the time-series data in ascending or descending order based on a time at which a biosignal is measured. As another example, the electronic apparatus 110 may sort each row of the time-series data in ascending or descending order based on a type of the biosignal. Through this, the electronic apparatus 110 may facilitate interpretation, storage, processing, and application of the time-series data by the language model.

According to an example embodiment, the electronic apparatus 110 may acquire the time-series data which indicates, for each of the respective types of the biosignals, the time at which the biosignal is measured and a value of the biosignal. This may list values of the biosignals according to a time measured for each of the respective types of the biosignals. The electronic apparatus 110 may convert pieces of data having the same measured time into one sentence in the time-series data. For example, the electronic apparatus 110 may convert biosignals measured 0.73 hours before the current time into one sentence that is “LACTATE is measured at 0.7000, PH is measured at 7.4250, HEMATOCRIT measured at 28.0000, and HCO3 measured at 21.3000 0.73 hours ago,” without separating the biosignals into multiple sentences. Considering that predetermined biosignals are collected through one test or measurement, the electronic apparatus 110 may prevent the time-series data from becoming unnecessarily redundant and efficiently manage capacity and volume of the time-series data. In addition, when an operation of sorting the time-series data in chronological order of measuring the biosignals precedes, such a sentence integration operation may be performed more easily.

In an example embodiment, when there are two or more pieces of data having the same type among the acquired time-series data, the electronic apparatus 110 may perform a preprocessing operation on the pieces of data having the same type before inputting the pieces of data to the input format A. For example, the electronic apparatus 110 may delete remaining data except for one most recently measured among the pieces of data having the same type. As another example, the electronic apparatus 110 may convert the pieces of data having the same type into one sentence and input the sentence to the input format A. In this case, the language model may easily grasp progress of a predetermined biosignal over time. As another example, for biodata types predetermined to require consideration of a time-series change pattern of a value of biodata in order to output the prediction result by using a prediction model among types of the biodata (such as electrocardiogram (ECG), heart rate variability (HRV), blood pressure, pulse, respiration, blood sugar, or temperature), the electronic apparatus 110 may perform interpolation as preprocessing based on a value of a measured biosignal.

Meanwhile, the electronic apparatus 110 may input the prediction result output by the prediction model to the input format A together with the time-series data. An example of the prediction result illustrated in FIG. 3 is a major adverse events score (MAES) which indicates, as a result of analysis by the prediction model based on the time-series data, a risk that an acute major adverse event occurs to a patient within a predetermined time.

Meanwhile, the electronic apparatus 110 may input the explanation data which is output by an explanation model to the input format A together with the time-series data and the prediction result. In FIG. 3, the explanation model may output, in a form of time-series data, a contribution (value) of each of pulse, respiration (RESP), systolic blood pressure (SBP), diastolic blood pressure (DBP), and temperature (TEMP) to the prediction result over time, and the electronic apparatus 110 may input the contribution (value) as the explanation data to the language model as an element of the input data. In an example embodiment, the electronic apparatus 110 may utilize the explanation data output by the explanation model directly as the input data, or when the explanation data is written in a format other than a preset format, the electronic apparatus 110 may convert the explanation data into the preset format (e.g., data formed with a numerical value and text) and utilize the converted explanation data as the input data. For example, the electronic apparatus 110 may convert the explanation data in a form of a graph of FIG. 3 into tabular data in which contributions (values) are individually matched to the biosignals over time.

Meanwhile, in FIG. 3, a decrease of PULSE from 72 to 48 at 0.0 hours before (that is, immediately before generation of the time-series data) is identified as a reasoning basis of the prediction model because the contribution (value) of PULSE at 0.0 hours to prediction of the MAES as 80 points is determined to be largest on the explanation data.

According to an example embodiment, with respect to prediction of the MAES as 80 points, the same biosignal may change at multiple time points, and a variety of natural language text data may be generated based thereon. Also, even at the same time point, multiple biosignals may change, and a variety of natural language text data may be generated based thereon. As such, a plurality of pieces of natural language text data may exist for each time point or biosignal corresponding to one prediction result, and the electronic apparatus 110 may select predetermined natural language text data from the plurality of pieces of natural language text data and provide the selected natural language text data to the user terminal 130. Criteria for the electronic apparatus 110 to select the natural language text data may be variously set. As an example, the electronic apparatus 110 may select up-to-date natural language text data from the plurality of pieces of natural language text data which is distinguished according to the time points. As another example, the electronic apparatus 110 may select natural language text data including a largest number of biosignals having a relatively high input attribution score based on the explanation data from the plurality of pieces of natural language text data which is distinguished according to the biosignals. As still another example, the electronic apparatus 110 may select, from the plurality of pieces of natural language text data which is distinguished by according to the time points and the biosignals, the natural language text data including the largest number of biosignals having the relatively high input attribution score based on the explanation data as a first priority and the up-to-date natural language text data as a second priority and provide both to the user terminal 130.

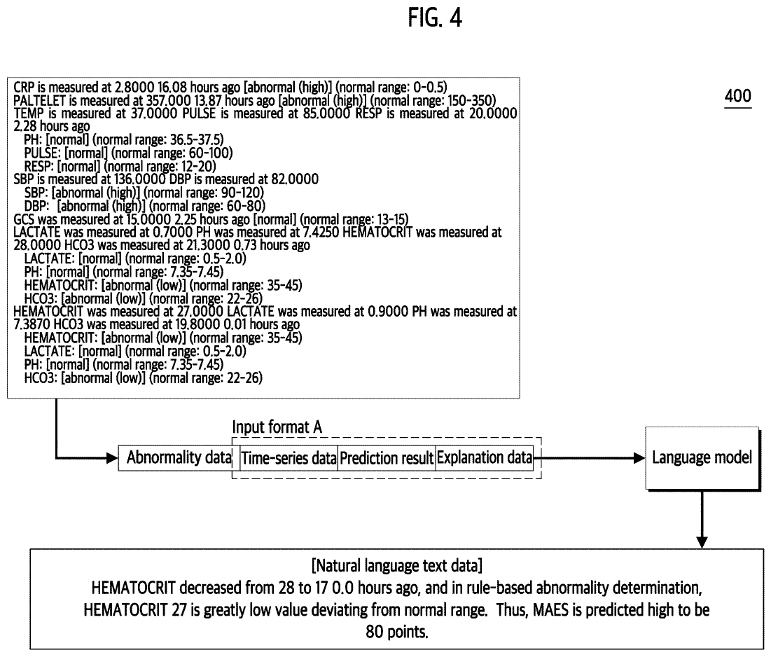

Next, FIG. 4 describes an example in which the input data which includes abnormality data, the time-series data, the prediction result, and the explanation data is input to the language model and the natural language text data is output.

According to an example embodiment, the abnormality data (or anomaly data or anomality data) may include whether at least some biosignals among a plurality of biosignals forming the time-series data is normal or abnormal, a normal range value for the at least some biosignals, and a class value classifying an abnormality of a biosignal determined to be abnormal.

According to an example embodiment, the electronic apparatus 110 may acquire the abnormality data in which an abnormality of the at least some biosignals among the plurality of biosignals forming the time-series data is evaluated based on a rule. Thereafter, the electronic apparatus 110 may input the input data including the abnormality data, the time-series data, the prediction result, and the explanation data to the language model.

The natural language text data output from the language model may include, as the reasoning basis of the prediction model, at least a portion of a biosignal identified by the explanation data or the biosignal determined to be abnormal according to the abnormality data among the plurality of biosignals forming the time-series data. As an example, the natural language text data may include a biosignal identified by the explanation data and simultaneously determined to be abnormal according to the abnormality data as a first-priority reasoning basis of the prediction model, a biosignal identified by the explanation data but not determined to be abnormal according to the abnormality data as a second-priority reasoning basis of the prediction model, and a biosignal determined to be abnormal according to the abnormality data but not identified by the explanation data as a third-priority reasoning basis of the prediction model.

As another example, the natural language text data may include, as the reasoning basis of the prediction model, a biosignal corresponding to a class with a high degree of abnormality among biosignals determined to be abnormal according to the abnormality data. Furthermore, when a plurality of biosignals corresponding to the class with the high degree of abnormality is present, the natural language text data may include, as the reasoning basis of the prediction model, a biosignal that deviates most from the normal range value among such a plurality of biosignals. At this point, a degree to which the biosignal deviates from the normal range value may be calculated as an absolute value or as a relative value of a deviating value compared to a normal range. For example, in FIG. 4, biosignals determined to have the ‘high’ degree of abnormality are C-reactive protein (CRP), PLATELET, the SBP, and the DBP, and the natural language text data may include the CRP, which has a relatively largest degree of deviation compared to the normal range among the biosignals, as the reasoning basis of the prediction model.

Meanwhile, in FIG. 4, HEMATOCRIT is identified as the reasoning basis of the prediction model according to rule-based abnormality determination. Although corresponding to a class with a low degree of abnormality, the HEMATOCRIT may be identified as the reasoning basis of the prediction model due to a large influence thereof on the prediction of the MAES according to a predefined rule.

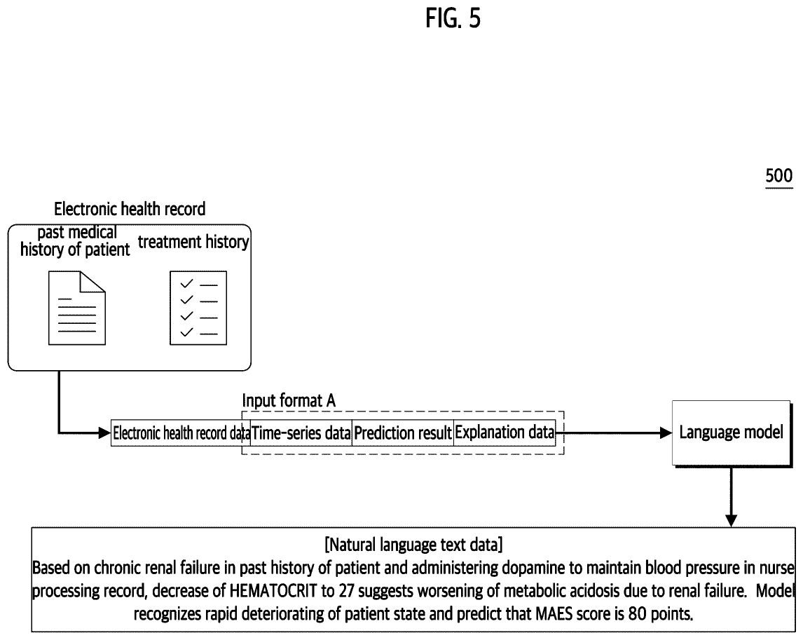

Next, FIG. 5 describes an example in which the input data which includes electronic health record data, the time-series data, the prediction result, and the explanation data is input to the language model, and the natural language text data is output.

According to an example embodiment, the electronic apparatus 110 may acquire the electronic health record (EHR) data including a past medical history and a treatment history of a patient. Thereafter, the electronic apparatus 110 may input the input data including the EHR data, the time-series data, the prediction result, and the explanation data to the language model.

The natural language text data output from the language model may include, as the reasoning basis of the prediction model, at least a portion of the biosignal identified by the explanation data and a biosignal identified by the EHR data among the plurality of biosignals forming the time-series data. For example, the natural language text data may include a biosignal identified by the explanation data and simultaneously identified by the EHR data as the first-priority reasoning basis of the prediction model, a biosignal identified by the explanation data but not identified by the EHR data as the second-priority reasoning basis of the prediction model, and a biosignal identified by the EHR data but not identified by the explanation data as the third-priority reasoning basis of the prediction model.

Meanwhile, when a plurality of biosignals identified by the EHR data is present, the natural language text data may include, as the reasoning basis of the prediction model, a biosignal most frequently identified according to the EHR data or a biosignal most recently identified by the EHR data.

Meanwhile, when the biosignal identified by the EHR data corresponds to the reasoning basis of the prediction model, the natural language text data may further include text information that explains the past medical history and the treatment history of the patient as a basis for determining a symptom of the patient and explains a diagnosis content corresponding to the biosignal identified by the EHR data as a result of determining the symptom of the patient. In FIG. 5, referring to the past medical history and treatment history of the patient as the EHR data, chronic renal failure of the patient in the past medical history and a record of administering dopamine to maintain blood pressure in a processing record of a nurse are explained as a ‘basis for determination’ in the natural language text data. Also, according to the basis for determination, that a decrease of the HEMATOCRIT to 27 suggests worsening of metabolic acidosis due to renal failure is explained as a ‘result of determination’. According to an example embodiment associated therewith, when a past medical history of the patient is identified in an EHR, a corresponding treatment history also may be identified, and a diagnosis content may be generated based on the identified medical history and treatment history. Generation of the diagnosis content may be performed based on a rule, but a pre-trained language model may also generate the diagnosis content by receiving the medical history and treatment history as an input.

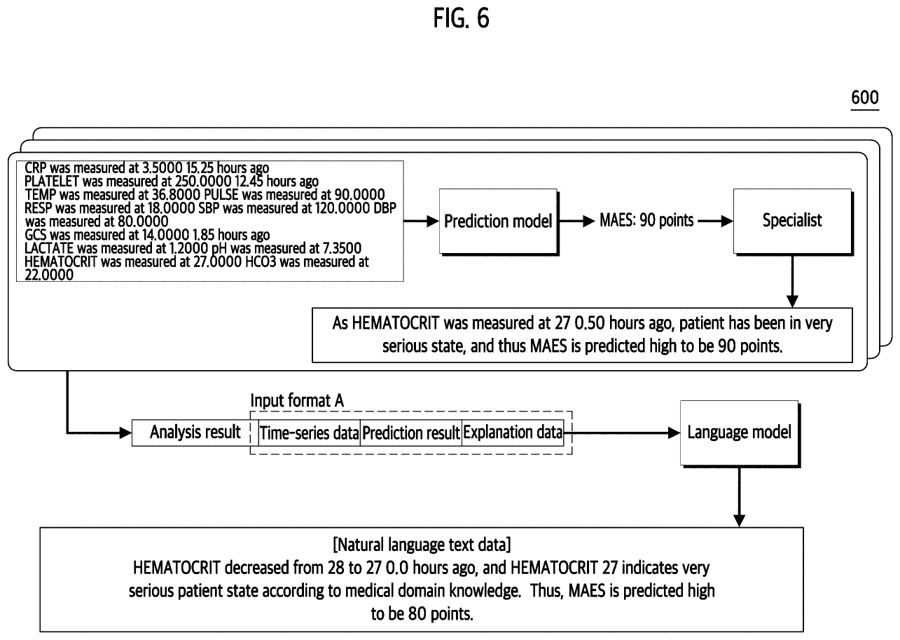

Next, FIG. 6 describes an example in which the input data which includes an analysis result by a specialist, the time-series data, the prediction result, and the explanation data is input to the language model, and the natural language text data is output.

According to an example embodiment, the electronic apparatus 110 may acquire the analysis result by the specialist on the time-series data and the prediction result. Thereafter, the electronic apparatus 110 may input the input data including the analysis result by the specialist, the time-series data, the prediction result, and the explanation data to the language model. According to an example embodiment, a format of the input data including the analysis result by the specialist, the time-series data, the prediction result, and the explanation data may have various forms such as text and an image. According to an example embodiment, the electronic apparatus 110 may acquire the input data including the analysis result by the specialist, the time-series data, the prediction result, and the explanation data from various electronic apparatuses including the user terminal 130.

The natural language text data output from the language model may include, as the reasoning basis of the prediction model, at least a portion of the biosignal identified by the explanation data or a biosignal identified by the analysis result by the specialist among the plurality of biosignals forming the time-series data. For example, the natural language text data may include a biosignal identified by the explanation data and simultaneously identified by the analysis result by the specialist as the first-priority reasoning basis of the prediction model, a biosignal identified by the explanation data but not identified by the analysis result by the specialist as the second-priority reasoning basis of the prediction model, and a biosignal identified by the analysis result by the specialist but not identified by the explanation data as the third-priority reasoning basis of the prediction model.

According to an example embodiment, the language model may be trained in advance with few-shot learning by the analysis result which includes a specialist opinion. That is, although FIG. 6 illustrates that the analysis result is input together with the time-series data, the prediction result, and the explanation data in reasoning of the language model, according to an example embodiment, the analysis result may be data that is input only in a learning process of the language model. Since labeling the specialist opinion for several cases in which the time-series data and the prediction result are matched requires significant cost and time, the language model may generate the natural language text data including the reasoning basis of the prediction model with high accuracy for a variety of input data by using only a small number of analysis results including the specialist opinion through a few-shot learning technique.

In FIG. 6, the HEMATOCRIT decreased from 28 to 27 at 0.0 hours, and this is identified as the reasoning basis of the prediction model because the above-described decrease indicates a highly serious patient state according to the specialist opinion (namely, medical domain knowledge) which states that “As HEMATOCRIT was measured at 27 0.50 hours ago, the patient has been in a very serious state, and thus the MAES is predicted high to be 90 points.” However, in identifying the reasoning basis of the prediction model, the language model does not consider only an analysis result for one case, and depending on example embodiments, may consider one or more cases for a biosignal that is the same as or highly associated with the biosignal identified by the explanation data. For example, when the biosignal identified by the explanation data is the CRP, the language model may refer to the specialist opinion on the CRP and one biosignal most associated with CRP. At this point, a relationship between biosignals may be defined in a table form in advance and stored in the database 120 or a storage medium linked with the electronic apparatus 110.

As such, FIGS. 4 through 6 individually describe example embodiments in which the abnormality data, the electronic health record data, and information on the specialist opinion is added as the input data to the language model and utilized as the reasoning basis for the prediction result of the prediction model. In addition to example embodiments of FIGS. 4 through 6, the electronic apparatus 110 may be implemented so that the language model outputs information on the reasoning basis for the prediction result of the prediction model in output data by combining at least two or more of the abnormality data, the electronic health record data, and the information on the specialist opinion as the input data and inputting the input data to the language model.

According to example embodiments, a case in which two or more of the abnormality data, the electronic health record data, and the information on the specialist opinion are added as the input data to the input format A may be considered.

For example, the electronic apparatus 110 may consider the abnormality data as a highest priority among the three elements described above, the electronic health record data as a second priority, and the information on the specialist opinion as a third priority. Accordingly, the electronic apparatus 110 may include, as the reasoning basis of the prediction model, a biosignal that is redundantly identified by the abnormality data among biosignals identified by the explanation data. However, when a plurality of biosignals redundantly identified by the explanation data and the abnormality data is present, the electronic apparatus 110 may include, as the reasoning basis of the prediction model, a biosignal redundantly identified by the electronic health record data among the plurality of redundantly identified biosignals. When a plurality of still identified biosignals is present, the electronic apparatus 110 may include, as the reasoning basis of the prediction model, a biosignal redundantly identified by the information on the specialist opinion. When a biosignal redundantly identified by first-priority and second-priority elements is absent, or when a biosignal redundantly identified by first-priority to third-priority elements is absent, all biosignals identified by an element having the highest priority (for example, a case in which a plurality of biosignals is present may be included) may be included as the reasoning basis of the prediction model.

Meanwhile, as another example, when two or more of the three elements described above are taken into consideration, the electronic apparatus 110 may determine which element is more important to the language model and input, separately from the input data, a prompt requesting to preferentially consider the element.

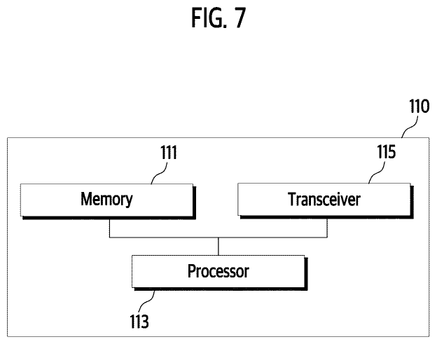

FIG. 7 is a block diagram illustrating the electronic apparatus 100 according to various example embodiments.

In an example embodiment, the electronic apparatus 110 may include a memory 111, a processor 113, and a transceiver 115. The electronic apparatus 110 may exchange data with an external entity through the transceiver 115 configured to communicate with a server or a client.

The processor 113 may perform at least one method described above through the drawings or support an external apparatus so that the external apparatus performs the method. The memory 111 may store information for performing at least one method described above through the drawings. The memory 111 may be a volatile memory or a non-volatile memory.

The processor 113 may execute a program and control the electronic apparatus 110 in order to provide information. Code of the program executed by the processor 113 may be stored in the memory 111.

According to an example embodiment, the processor 113 may be connected to the memory 111 and the transceiver 115 to acquire time-series data on a patient, acquire, from an artificial intelligence-based prediction model, a prediction result that is output by the prediction model based on the time-series data, acquire, from an explainable artificial intelligence-based explanation model, explanation data that is output by the explanation model based on the time-series data and the prediction result, input input data including the time-series data, the prediction result, and the explanation data to an artificial intelligence-based language model, and acquire, from the language model, natural language text data that is output by the language model based on the input data and includes a reasoning basis of the prediction model for the prediction result.

Elements only associated with the present example embodiment are illustrated for the electronic apparatus 110 illustrated in FIG. 7. Therefore, those skilled in the art related to the present example embodiment will understand that other general-purpose elements may be further included in addition to the elements illustrated in FIG. 7.

The apparatus according to the above-described example embodiments may include a processor, a memory for storing and executing program data, a permanent storage such as a disk drive, a communication port for communicating with an external device, a user interface device such as a touch panel, a key, or a button, or the like. Methods implemented with a software module or an algorithm may be stored in a computer-readable recording medium as computer-readable codes or program instructions executable on the processor. Here, the computer-readable recording medium includes magnetic storage media (e.g., a read-only memory (ROM), a random-access memory (RAM), a floppy disk, a hard disk, or the like.), optical reading media (e.g., a compact-disc read-only memory (CD-ROM) or a digital versatile disc (DVD)), or the like. The computer-readable recording medium may be distributed over network-connected computer systems so that the computer-readable codes are stored and executed in a distributed manner. The medium may be readable by a computer, stored in a memory, and executed in a processor.

The present example embodiment may be represented as functional block configurations and various processing steps. Such functional blocks may be implemented with various numbers of hardware or/and software configurations that execute predetermined functions. For example, the example embodiment may employ integrated circuit configurations such as a memory, processing, logic, and a look-up table that may execute various functions under control by one or more microprocessors or other control devices. Similarly to the execution of elements as software programming or software elements, the present example embodiment may be implemented in a programming or scripting language such as C, C++, Java, or assembler language, including various algorithms implemented as a combination of data structures, processes, routines, or other programming configurations. Functional aspects may be implemented as an algorithm executed on one or more processors. Also, the present example embodiment may adopt conventional technology for electronic configuration, signal processing, message processing, data processing, and/or the like. Terms such as “mechanism,” “element,” “means,” and “configuration” may be used broadly and are not limited to mechanical and physical configurations. The terms may include the meaning of a series of routines of software in association with a processor or the like.

The above-described example embodiments may be implemented with artificial intelligence (AI) through a processor and a memory of an electronic apparatus. The processor may be formed with one or a plurality of processors, and the one or the plurality of processors may be a general-purpose processor such as a central processing unit (CPU), an application processor (AP), or a digital signal processor (DSP), a graphics-only processor such as a graphics processing unit (GPU) or a vision processing unit (VPU), or an artificial intelligence-only processor such as a neural processing unit (NPU). The one or the plurality of processors may control to process input data according to a predefined operation rule or an artificial intelligence model stored in the memory. Alternatively, when the one or the plurality of processors are artificial intelligence-only processors, the artificial intelligence-only processor may be designed with a hardware structure specialized for processing a predetermined artificial intelligence model.

The predefined operation rule or the artificial intelligence model is characterized by being created through learning. Here, being created through learning refers to that the predefined operation rule or the artificial intelligence model which is set to perform desired characteristics (or purposes) is created as a basic artificial intelligence model is trained by using multiple pieces of learning data by a learning algorithm. Such learning may be performed in the electronic apparatus itself in which the artificial intelligence according to the present disclosure is performed or may be performed through a separate server and/or system. Examples of the learning algorithm include supervised learning, unsupervised learning, semi-supervised learning, or reinforcement learning, but are not limited to the above-described examples.

The artificial intelligence model may be formed with a plurality of neural network layers. Each of the plurality of neural network layers has a plurality of weight values and may perform a neural network operation through an operation between an operation result of a previous layer and the plurality of weight values. The plurality of weight values of the plurality of neural network layers may be optimized by a learning result of the artificial intelligence model. For example, the plurality of weight values may be updated so that a loss value or a cost value acquired from the artificial intelligence model in a learning process is reduced or minimized. An artificial neural network may include a deep neural network (DNN), a convolutional neural network (CNN), a recurrent neural network (RNN), a restricted Boltzmann machine (RBM), a deep belief network (DBN), a bidirectional recurrent deep neural network (BRDNN), Deep Q-Networks, or the like, but is not limited to the above-described examples.

The above-described example embodiments are merely an example, and other example embodiments may be implemented within the scope of the claims described below.

What is claimed is:

1. A method for providing an explanation for patient state prediction through an electronic apparatus, the method comprising:
acquiring time-series data on a patient;
acquiring, from an artificial intelligence-based prediction model, a prediction result that is output by the prediction model based on the time-series data;
acquiring, from an explainable artificial intelligence-based explanation model, explanation data that is output by the explanation model based on the time-series data and the prediction result;
acquiring abnormality data in which an abnormality of at least a portion of biosignals among a plurality of biosignals forming the time-series data is evaluated based on a rule;
inputting input data including the time-series data, the prediction result, the explanation data, and the abnormality data to an artificial intelligence-based language model; and
acquiring, from the language model, natural language text data that is output by the language model based on the input data and includes a reasoning basis of the prediction model for the prediction result,
wherein the natural language text data includes, as the reasoning basis of the prediction model, at least a portion of a biosignal identified by the explanation data and a biosignal determined to be abnormal according to the abnormality data among the plurality of biosignals forming the time-series data.

2. The method of claim 1, wherein the time-series data includes information on a time at which a biosignal of the patient is measured, a value of the biosignal, and a type of the biosignal.

3. The method of claim 1, wherein the prediction result includes at least a portion of a sepsis score, an acute major adverse events score, and an acute mortality score of the patient.

4. The method of claim 1, wherein the explanation data includes an input attribution score of the time-series data for the prediction result.

5. The method of claim 1, wherein the inputting comprises generating input data corresponding to an input format of the language model by concatenating the time-series data, the prediction result, the explanation data, and the abnormality data.

6. The method of claim 1, wherein the natural language text data includes, as the reasoning basis of the prediction model, a biosignal identified by the explanation data among a plurality of biosignals forming the time-series data.

7. The method of claim 1, wherein the abnormality data includes:
whether the at least a portion of biosignals among the plurality of biosignals forming the time-series data is normal or abnormal;
a normal range value for the at least a portion of biosignals; and
a class value classifying an abnormality of a biosignal determined to be abnormal.

8. The method of claim 1, wherein the natural language text data includes:
a biosignal identified by the explanation data and simultaneously determined to be abnormal according to the abnormality data as a first-priority reasoning basis of the prediction model;
a biosignal identified by the explanation data and not determined to be abnormal according to the abnormality data as a second-priority reasoning basis of the prediction model; and
a biosignal determined to be abnormal according to the abnormality data and not identified by the explanation data as a third-priority reasoning basis of the prediction model.

9. The method of claim 1, further comprising acquiring electronic health record (EHR) data including a past medical history and a treatment history of the patient,
wherein the input data further includes the EHR data, and
the natural language text data includes, as the reasoning basis of the prediction model, at least a portion of the biosignal identified by the explanation data, the biosignal determined to be abnormal according to the abnormality data, and a biosignal identified by the EHR data among the plurality of biosignals forming the time-series data.

10. The method of claim 9, wherein when the biosignal identified by the EHR data corresponds to the reasoning basis of the prediction model, the natural language text data further includes text information that explains the past medical history and the treatment history of the patient as a basis for determining a symptom of the patient and explains a diagnosis content corresponding to the biosignal identified by the EHR data as a result of determining the symptom of the patient.

11. The method of claim 1, further comprising acquiring a result of analysis by a specialist on the time-series data and the prediction result,
wherein the input data further includes the result of analysis by the specialist, and
the natural language text data includes, as the reasoning basis of the prediction model, at least a portion of the biosignal identified by the explanation data, the biosignal determined to be abnormal according to the abnormality data, and the biosignal identified by the result of analysis by the specialist among a plurality of biosignals forming the time-series data.

12. A non-transitory computer-readable recording medium storing a program configured to cause a computer to execute the method of claim 1.

13. An electronic apparatus for providing an explanation for patient state prediction, the electronic apparatus comprising:
a transceiver configured to communicate with an external entity;
a memory configured to store an instruction; and
a processor,
wherein the processor is configured to control the transceiver and the memory to:
acquire time-series data on a patient;
acquire, from an artificial intelligence-based prediction model, a prediction result that is output by the prediction model based on the time-series data;
acquire, from an explainable artificial intelligence-based explanation model, explanation data that is output by the explanation model based on the time-series data and the prediction result;
acquire abnormality data in which an abnormality of at least a portion of biosignals among a plurality of biosignals forming the time-series data is evaluated based on a rule;
input input data including the time-series data, the prediction result, the explanation data, and the abnormality data to an artificial intelligence-based language model; and
acquire, from the language model, natural language text data that is output by the language model based on the input data and includes a reasoning basis of the prediction model for the prediction result,
wherein the natural language text data includes, as the reasoning basis of the prediction model, at least a portion of a biosignal identified by the explanation data and a biosignal determined to be abnormal according to the abnormality data among the plurality of biosignals forming the time-series data.

14. A system for providing an explanation for patient state prediction to a user terminal, the system comprising:
an electronic device; and
the user terminal configured to receive and display the natural language text data,
wherein the electronic device is configured to:
acquire time-series data on a patient;
acquire, from an artificial intelligence-based prediction model, a prediction result that is output by the prediction model based on the time-series data;
acquire, from an explainable artificial intelligence-based explanation model, explanation data that is output by the explanation model based on the time-series data and the prediction result;
acquire abnormality data in which an abnormality of at least a portion of biosignals among a plurality of biosignals forming the time-series data is evaluated based on a rule;
input input data including the time-series data, the prediction result, the explanation data, and the abnormality data to an artificial intelligence-based language model; and
acquire, from the language model, natural language text data that is output by the language model based on the input data and includes a reasoning basis of the prediction model for the prediction result,
wherein the natural language text data includes, as the reasoning basis of the prediction model, at least a portion of a biosignal identified by the explanation data and a biosignal determined to be abnormal according to the abnormality data among the plurality of biosignals forming the time-series data.
